(19) United States
(12) Patent Application Publication
(10) Pub. No.: US 2022/0080033 A1
(43) Pub. Date: Mar. 17, 2022

(54) A LYOPHILIZED COMPOSITION OF PEGASPARGASE

(71) Applicant: Gennova Biopharmaceuticals Limited, Maharastra (IN)

(72) Inventors: Tathagata Mukherjee, Maharastra (IN); Praveen Kumar Agarwal, Maharastra (IN); Sanjay Singh, Maharastra (IN)

(21) Appl. No.: 17/414,790

(22) PCT Filed: May 20, 2019
(86) PCT No.: PCT/IN2019/050402

§ 371 Date: Jun. 16, 2021

(30) Foreign Application Priority Data
Dec. 24, 2018 (IN) 201821048859

(51) Int. Cl. A61K 38/50; A61K 47/02; A61K 47/26; A61K 9/19; A61K 47/60

(52) U.S. Cl. CPC A61K 38/50; A61K 47/02; C12Y 305/01001; A61K 9/19; A61K 47/60; A61K 47/26

(57) ABSTRACT

The present invention relates to a novel, economically viable, storage stable, lyophilized composition of pegaspargase. The composition comprises pegaspargase, a cryoprotectant, a bulking agent, a buffer and may optionally contain other pharmaceutically acceptable excipients including but not limited to a salt. The composition of the present invention is stable for extended periods over significant range of temperatures, without the presence of any significant amount of impurities. The present invention also relates to an economically viable and scalable lyophilization process for the production of the storage stable composition of pegaspargase.

### CROSS-REFERENCE TO RELATED APPLICATIONS

The present invention is the US National Stage under 35 U.S.C. § 371 of International Application No. PCT/IN2019/050402, having a filing date of May 20, 2019, which claims benefit of priority from Indian Application No. 201821048859, filed Dec. 24, 2018. The disclosures of the prior applications are considered part of (and are incorporated by reference in) the disclosure of this application.

### BACKGROUND OF THE INVENTION

The present invention relates to the field of biopharmaceutical sciences. In particular, it relates to a lyophilized composition of pegaspargase and process for the preparation of the same.

Protein drug delivery remains a major challenge for the biopharmaceutical industry because of the inherent instability of proteins exhibited in vivo. While proteins administered orally are susceptible to their digestion in the digestive tract, proteins injected parenterally are generally found prone to renal clearance and proteolysis. Other problems that are typically associated with protein drugs are low solubility, short circulating half-life, immunogenicity, aggregation etc. As a result, sustainability of the protein in the body is compromised. Several approaches to achieve sustainability of proteins in vivo have been tried out such as alteration of amino acid sequence to decrease immunogenicity and eliminate proteolytic cleavage sites, conjugation of proteins to serum proteins, fusion with antibodies, incorporation into liposomes for slow release, conjugating with natural or synthetic polymers etc.

Conjugation of therapeutic proteins with polymers such as polyethylene glycol (PEG) has been used in biopharmaceutical industries successfully for a long time and is accepted as a safe modification of proteins for increasing circulating half-life, reducing immunogenicity, and a disclosure in this regard may be found in U.S. Pat. No. 4,179,337. This process of conjugation is termed as pegylation. PEG has been categorized by the regulatory bodies such as USFDA and WHO under the compounds ‘Generally Recognized as Safe’ (GRAS).

PEG is a linear or branched polymer and is water soluble (solubility increases with increasing molecular weight), lipophilic and nontoxic. The lipophilic property of PEG makes it amenable to end group functionalization for ready conjugation to therapeutic proteins. Each molecule of PEG typically binds 2-3 water molecules per ethylene oxide unit. Pegylation, thus mask the protein's surface and increases the molecular size of the polypeptide, thus preventing the access of antibodies or antigen processing cells and also reduces the degradation by proteolytic enzymes, which results in increased circulatory half-life. Further, the increase in size (due to increase of the hydrodynamic radii) prolongs its circulatory time by reducing renal clearance.

In many type of cancerous cells like those involved in acute lymphoblastic leukemia (ALL), the cancerous cells are not able to synthesize the amino acid L-asparagine de novo (because they lack or have low levels of the enzyme asparagine synthetase, which catalyzes the enzymatic transformation of the amino acid L-aspartate to L-asparagine) and takes it up from blood for cell growth. L-asparaginase is an enzyme that catalyzes the hydrolysis of L-asparagine to L-aspartate with the release of ammonia. L-Asparaginase depletes the L-asparagine levels from the blood thereby preventing its uptake by the cancer/tumor cells ultimately leading to the death of the cells. L-Asparaginase may be obtained from several sources including bacterial, yeast, fungi, actinomycetes and plants. It is useful in treating tumors or cancers that are dependent upon L-asparagine for protein synthesis. It is particularly used for the treatment of leukemias, such as acute lymphoblastic leukemia and is typically used in combination with other anti-tumor or anticancer therapies, although it can be employed alone in certain clinical situations.

However, L-asparaginase itself, suffers from the usual disadvantages of being a protein, such as high rate of clearance, short half-life, proteolytic degradation, and the potential for inducing an immune response due to non-human origin in patients treated with this enzyme. These shortcomings limits the use of this enzyme for longer treatment or repeated dosing. As discussed earlier these problems can be overcome by pegylation. L-Asparaginase (from E. coli) is modified by covalently conjugating it with 5 kDa monomethoxypolyethylene glycol (mPEG). The resultant pegaspargase has the advantages of being substantially non-antigenic and exhibits a reduced rate of clearance from the circulation.

Pegaspargase, generally presented as a liquid composition in 5 mL pack size of concentration 750 IU/mL, was initially approved for the indication of acute lymphoblastic leukemia in patients who have developed hypersensitivity to the native forms of L-asparaginase, by the US-FDA in 1994 and was commercialized with the brand name Oncaspar®. Later, in 2006, Oncaspar® got approval for the first-line treatment of patients with acute lymphoblastic leukemia (ALL) as a component of a multiagent chemotherapy regimen. Oncaspar® was manufactured by pegylation of a 5 kDa monomethoxypolyethylene glycol (mPEG) Succinimidyl Succinate PEG (also referred to SS-PEG). Pegylated asparaginase is disclosed in U.S. Pat. Nos. 5,122,614; 5,324,844; 5,612,460; US20120100121A1; CN105802946A applications.

Despite having all the advantages, the liquid composition of pegaspargase has been reported to have problems such as thermal stability, strict requirement for cold—chain maintenance, shorter shelf-life etc. It has been reported that pegylated proteins, especially those that are linked with succinate linker tend to degrade in its liquid composition to result in free PEG and succinylated protein as a result of hydrolysis of the ester linkage between the PEG and the succinate linker in aqueous composition. Access of such critical drugs in clinical practice require compositions that can be stored for an extended period which can also sustain temperature excursions during manufacturing and while distribution to clinics.

More often than not the stability issues associated with the proteins in liquid composition can be overcome by presenting it in solid composition. Removal of water is often proved effective since all the major degradation reaction (deamidation, hydrolysis, proteolysis etc.) occur in the aqueous solution. The most prevalent method used for liquid to solid transformation is freeze-drying or lyophilization. Lyophilization is a process which can help stabilize the pegaspargase and overcome the challenges. Over half of the therapeutics of biological origin that are commercialized are presented as lyophilized composition.

Lyophilization cycle consists of primarily three steps: freezing, primary drying and secondary drying with an optional annealing step between freezing and primary drying. The process of lyophilization is not stress free and does not always guarantee an extended shelf-life of the biopharmaceutical product. The stress associated with the lyophilization steps can cause both physical (denaturation, aggregation, precipitation etc.) as well as chemical degradations (oxidation, Maillard reaction, covalent aggregation etc.) of the protein. These degradative pathways which eventually leads to loss of its bio-activity and are not mutually exclusive as often one leads to another and both the degradative pathways are somewhat linked.

Design of a lyophilization cycle depends on the concentration of the protein, nature and the amount of bulking agents, stabilizers and other excipients present in the composition. Important thermal parameters like the apparent glass transition temperature (Tg′), crystallization temperature of the bulking agent etc. are usually determined for the compositions prior to the design of the process as they serve guidance point for setting up the temperature and pressure parameters for each step including the ramp and holding time at each stage of the lyophilization cycle.

Pegylated proteins present other complexities that needs to be addressed for determining the final lyophilization process such as the state of the PEG, being amorphous or crystalline, amount of free water available for interaction, storage temperature, lyophilization parameters, ratio of protein to PEG, all of which influences the activity of the freeze dried protein post lyophilization, and there is no universal solution for all pegylated products.

Hence, it is important to craft a unique process and composition for each protein as the process and composition suitable for one protein may not be effective for another.

U.S. Pat. Nos. 6,180,096 and 7,632,491 B2 discloses composition of pegylated interferon 2b with longer lyophilization cycle, high moisture content. U.S. Pat. No. 8,367,054 B2 discloses a composition for Peg-interferon 2b with a shorter lyophilization cycle. These documents disclose the importance of the lyophilization process and suggest that there is a change in the quality of the product based on the lyophilization cycle.

CN105796507A discloses a stable composition of pegaspargase containing sorbitol, a protective agent, a buffering agent and a surfactant. However, the composition addresses the stability in the liquid form and protection while freezing. The said application failed to provide a stable freeze-dried composition.

WO2018017190, discloses a lyophilized storage stable composition, the composition comprising a polyalkylene oxide-asparaginase comprising a polyalkylene oxide group covalently linked by a linker to L-asparaginase; a buffer; a salt; and a sugar.

However, the process as disclosed in WO′190 is long (˜5 days) and not economical. Moreover, it utilizes large quantities of excipients, which is not preferred, since it may increase the cost of excipient by ˜50% and thus the cost of final product.

Pegaspargase is categorized as an orphan drug and is highly priced. This, process of storage stable product preparation adds in the cost of lyophilization as well as the additional excipients which will make the product more costly.

Hence, there is a need for an optimum storage stable lyophilized composition of pegaspargase that maintains the physical property and biological activity during its shelf-life and a lyophilization process for such a composition.

### OBJECT OF THE INVENTION

An object of the present invention is to provide an optimum storage stable lyophilized composition comprising pegaspargase which exhibits physicochemical stability and biological activity during its shelf-life and a lyophilization process for such a composition.

### SUMMARY OF THE INVENTION

The present invention provides an optimum storage stable lyophilized composition comprising pegaspargase which exhibits physicochemical stability and biological activity during its shelf-life and a lyophilization process for such a composition.

The composition of the present invention is stable for extended periods over significant range of temperatures, without the presence of any significant amount of impurities/degradants. The present invention also relates to an economically viable and scalable lyophilization process for the production of the storage stable composition of pegaspargase.

### BRIEF DESCRIPTION OF FIGURES

FIG. 1 depicts cake structure for the compositions within the scope of the present invention.

FIG. 2 depicts the analytical data demonstrating the integrity and purity of pegaspargase, pre and the post lyophilization (FIG. 2(B)) as assessed by sodium dodecyl sulfate-polyacrylamide gel electrophoresis (SDS-PAGE) and size exclusion high-performance liquid chromatography (SE-HPLC) respectively.

FIG. 3 depicts the analytical data as assessed by sodium dodecyl sulfate-polyacrylamide gel electrophoresis (SDS-PAGE) comparing the stability of prior art liquid composition of pegaspargase (FIG. 3(A)) with lyophilized composition of the present invention (FIG. 3(B)).

FIG. 4 depicts the analytical data as assessed by sodium dodecyl sulfate-polyacrylamide gel electrophoresis (SDS-PAGE) demonstrating the presence of high molecular weight impurities present in the prior art lyophilized composition of pegaspargase with lyophilized composition of the present invention by Coomassie staining (FIG. 4(A)), iodine staining (FIG. 4(B)). FIG. 4(C) and FIG. 4(D) shows the Western Blot analysis of the samples against anti-asparaginase antibody and anti-PEG antibody respectively.

### DETAILED DESCRIPTION OF THE INVENTION

### Composition

The present invention provides an optimum storage stable lyophilized composition comprising pegaspargase which exhibits physicochemical stability and biological activity during its shelf-life and a lyophilization process for such a composition.

The lyophilized composition of the present invention comprises as the active ingredient pegaspargase. The lyophilized composition of the present invention comprises pegaspargase, a cryoprotectant, a bulking agent, a buffer and may optionally contain other pharmaceutically acceptable excipients including but not limited to a salt.

The lyophilized composition of the present invention comprises a pegylated asparaginase comprising a polyalkylene oxide group covalently linked by a linker to asparaginase.

The composition of the present invention is drawn to pegylated asparaginase. Pegylated asparaginase also known as pegaspargase comprises a mono-methoxy polyethylene glycol (mPEG) of molecular weight preferably between 4-6 kDa, more preferably between 4.5-5.5 kDa and most preferably 4.8-5.2 kDa that is covalently linked by a succinate linker via an amide bond to one or more primary amine groups (terminal amine and ε-amino acid of lysine side chain) of L-asparaginase.

L-Asparaginase may be naturally obtained from E. coli or other bacterial sources like Erwinia chrysanthemi or through genetically engineering in E. coli through recombinant technology.

The conjugation reaction of mPEG and L-asparaginase results in covalent attachment of 1-12 mPEGs per monomer of L-asparaginase, preferably between 5-10 mPEGs per monomer of L-asparaginase, more preferably between 7-10 mPEGs per monomer of L-asparaginase and most preferably between 7-9 mPEGs per monomer of L-asparaginase.

The amount of pegaspargase of the invention may be present in a concentration (total weight percentage) of 2-32% of the composition; more preferably 5-20% and most preferably between 6-14%.

The process of lyophilization set out herein is novel and inventive in terms of the optimum amount of excipients utilized in the process. The excipients of the present invention, renders the composition of the present invention to be physico-chemically stable and biologically active during the shelf-life. The excipients also enable the design of a short and economic lyophilization cycle so as to obtain the product of the present invention. The composition of the present invention containing pegaspargase and excipients as set out herein is synergistic.

The composition of the present invention includes a cryoprotectant. The cryoprotectant may be selected from sugar, polyol, polymer, and amino acid. More preferably, the cryoprotectant of the present invention is sugar. Most preferably, the cryoprotectant is sucrose. The cryoprotectant may be present in a range of 9-91% of the composition; more preferably 20-60% and most preferably between 32-41% of the composition of the present invention. Without being limited by theory, the composition of the present invention envisages a cryoprotectant that may serve both as a cryo and lyoprotectant in order to reduce the burden of the excipients during the cycle. It may also serve as a stabilizer.

The composition of the present invention comprises a bulking agent. The bulking agent of the present invention is selected from the group comprising sugar, polyol, polymer, and amino acid, preferably the bulking agent is an amino acid selected from the group comprising glycine, histidine, arginine; preferably the amino acid is glycine. The bulking agent of the invention may be present in the range of 1-78% of the composition; more preferably 20-60% and most preferably between 38-50% of the composition of the present invention.

The composition of the present invention comprises a buffer. The buffer may be selected from the group comprising phosphate buffer such as sodium phosphate buffer (Sodium dihydrogen phosphate-disodium hydrogen phosphate) or potassium phosphate buffer (potassium dihydrogen phosphate-dipotassium hydrogen phosphate), TRIS, citrate buffer; preferably, the composition of the present invention comprises phosphate buffer. The pH of the product before lyophilization and after reconstitution of the lyophilized product may be 6-8. The buffer of the present invention may be present in the range of 3-33% of the composition; more preferably 3-15% and most preferably between 4-6% of the composition of the present invention.

The composition of the present invention may optionally comprise salt, selected from the group comprising sodium chloride, potassium chloride, preferably sodium chloride. The amount of the salt in the composition may be in the range of 0-40%, preferably 0-10%, more preferably 0-0.5% of the composition of the present invention. Without being limited by theory, the composition of the present invention is characterized in that it comprises low or no salt, i.e. the composition of the present invention may comprise a salt in a very low quantity unlike the compositions of the prior art and may also free of salt.

The composition of the present invention has osmolality preferably in the range of 250-600 mOsm/Kg, more preferably 250-500 mOsm/Kg and most preferably 250-450 mOsm/Kg.

### Lyophilization Process

It is submitted that the lyophilization processes are unique to the excipients and the active ingredient, and the processes have to be developed separately for each composition. Furthermore, processes in prior art are lengthy, uses high proportion of excipients and are not economical.

The lyophilized process of the present invention formulates the active pharmaceutical material in a manner that the lyophilized product obtained has the following characteristics:
a. Elegant cake that does not stick to the side of the vial
b. Consistent moisture content
c. Extended shelf-life (at room temperature)
d. Dissolves readily on reconstitution, produces a clear solution
e. Activity of the protein is intact
f. No alteration in the structure of the protein
g. pH is maintained
h. The reconstituted solution is within acceptable Osmolality range for parenteral administration

Lyophilization cycle consists of primarily three steps: freezing, primary drying and secondary drying with an optional annealing step between freezing and primary drying. Each of these step have been optimized for the composition of the present invention. In addition, it is envisaged that the process as set out herein will also be applicable to the similar compositions comprising pegaspargase as the active ingredient.

The total time of the lyophilization process in the present invention is preferably from 2880 min (48 h) to 5790 min (96.5 h), more preferably from 3120 min (52 h) to 4980 min (83 h), most preferably from 3120 min (52 h) to 4200 min (70 h). The lyophilized process in the present invention preferably includes variation of temperature from −60° C. to 30° C., more preferably from −50° C. to 30° C., most preferably from −40° C. to 25° C. The pressure variation of the lyophilization process in the present invention is preferably from 0.037 Torr to 760 Torr.

### Pre-Lyophilization

The concentration of pegaspargase present in the composition before lyophilization is preferably in the range of 4-25%, more preferably in the range of 6-20%, most preferably in the range of 8-16% of the composition.

The fill volume of before lyophilization is preferably in the range of 0.5 to 5 ml, more preferably in the range of 0.5 to 4 ml, most preferably in the range of 0.5 to 3 ml.

Based on the fill volume as well as the volume post reconstitution, appropriate concentration of additives (if any) needs to be added such that the desired concentration of the additives are obtained post reconstitution of the lyophilized product prior to its administration.

### Lyophilization Cycle

### Step—1—Freezing

The lyophilized process in the present invention preferably includes the lowest temperature of the freezing step from −10° C. to −60° C., more preferably from −20° C. to −50° C., most preferably from −35° C. to −45° C. The total time of the freezing step of the lyophilization process in the present invention is preferably from 150 min to 500 min, more preferably from 200 min to 400 min, most preferably from 240 min to 350 min. The time required to reach the lowest freezing temperature of the freezing step of the lyophilization process in the present invention is preferably from 20 min to 180 min, more preferably from 30 min to 120 min, most preferably from 45 min to 90 min. The hold time at the lowest freezing temperature of the freezing step of the lyophilization process in the present invention is preferably from 120 min to 480 min, more preferably from 250 min to 360 min, most preferably from 200 min to 300 min.

### Step—2—Primary Drying

The lyophilized process in the present invention preferably includes the starting temperature of the primary drying step from 10° C. to −50° C., more preferably from 0° C. to −45° C., most preferably from −30° C. to −40° C. The total time of the primary drying step of the lyophilization process in the present invention is preferably from 35-80 h, more preferably from 40-75 h, most preferably from 50-60 h. The time required to reach the starting temperature of the primary drying step of the lyophilization process in the present invention is preferably from 100 min to 1000 min, more preferably from 250 min to 500 min, most preferably from 300 min to 400 min. The pressure at the beginning of the primary drying step of the lyophilization process in the present invention is preferably from 50 mTorr to 200 mTorr. The maximum temperature at the end of the primary drying step of the lyophilization process in the present invention is preferably from 5° C. to 25° C., more preferably from 8° C. to 22° C., most preferably from 10° C. to 20° C. The hold time at the maximum temperature of the primary drying step of the lyophilization process in the present invention is preferably from 5-72 h, more preferably from 8-24 h, most preferably from 10-14 h. The pressure at the end of the primary drying step of the lyophilization process in the present invention is preferably from 37 mTorr to 112 mTorr, more preferably from 50 mTorr to 90 mTorr, most preferably from 60 mTorr to 80 mTorr.

The primary drying step of the lyophilization process in the present invention could also include one or more intermediate steps of drying. Temperature of the intermediate drying step of the lyophilization process in the present invention is preferably from −5° C. to 15° C., more preferably from 0° C. to 10° C., most preferably from 3° C. to 7° C. The hold time at the intermediate drying step of the lyophilization process in the present invention is preferably from 2-24 h, more preferably from 5-12 h, most preferably from 8-10 h. The pressure at the intermediate drying step of the lyophilization process in the present invention is preferably from 75 mTorr to 200 mTorr, most preferably from 100 mTorr to 120 mTorr.

### Step—3—Secondary Drying

At the end of the primary drying cycle the dried powder typically retains 10% of the moisture that needs to be removed by the incorporation of a secondary cycle. This is the last cycle of the lyophilization process and removes the unfrozen water i.e. the water associated with the amorphous state to further dry the product and reduce the residual moisture content.

The lyophilized process in the present invention preferably includes the temperature of the secondary drying step from 10° C. to 37° C., more preferably from 15° C. to 35° C., most preferably from 20° C. to 30° C. The total time of the secondary drying step of the lyophilization process in the present invention is preferably from 3-24 h, more preferably from 4-16 h, most preferably from 4-7 h. The hold time of the secondary drying step of the lyophilization process in the present invention is preferably from 3-24 h, more preferably from 4-16 h, most preferably from 4-7 h. The pressure of the secondary drying step of the lyophilization process in the present invention is preferably from 37 mTorr to 50 mTorr.

### Post-Lyophilization

The reconstitution volume per vial, post lyophilization can be 1-5.5 mL, depending on the desired dose post-reconstitution and the initial concentration of the pre-lyophilization sample. The concentration of pegaspargase after reconstitution of lyophilized product in the required volume is in the range of 750±20% IU/ml.

### Utility

In another aspect of the invention it is observed that the composition of the present invention is stable for extended periods in spite of temperature fluctuation that occurs during handling and transportation, since the product is stable at room temperature as well as 30° C. and 37° C. for substantial time interval. Without being limited by theory, it is proposed that the optimum use of the various ingredients at the said ratio maintains the stability of the composition during and after the lyophilization rendering a more stable product. The composition of the present invention permits to achieve a lyophilized product that maintains physical integrity, biological activity and chemical stability.

Further, the composition of the present invention contains pegaspargase which has purity greater than 95% post lyophilization. With this higher percentage purity, the composition is stabilized well, and the deterioration is found minimal at both accelerated and real time conditions of the stability.

The composition of the present invention is also synergistic in that the ingredients when constituted together as per the principles herein yield a composition having appropriate activity and stability during its shelf-life.

### Advantages of the Composition of the Present Invention

1. The composition of the present invention is such that it provides desired mechanical support to the cake structure thereby imparting stability to the composition of the pegaspargase and increase its ability to handle stress of the lyophilization process, resulting in a storage stable product.
2. The process of the present invention is such that the time of lyophilization is reduced considerably than other prior art processes, thereby paving way to an economically viable process with nearly 2 days of less run time of the lyophilizer and facility utilization. The lyophilization process optimized for the novel composition of pegaspargase as detailed in this invention is completed in less than 3 days which is a marked improvement over the nearly 5 days (112.5 hours) lyophilization process.
3. The composition of the present invention allows for the use of no or low salt concentration in the composition of lyophilized pegaspargase, providing advantages in terms of eutectic and glass transition temperature.
4. The novel composition mentioned in the present invention produces a storage stable product even at higher temperature. The stability of the lyophilized pegaspargase at 30° C. for 18 months and 37° C. for 3 months clearly demonstrates the improvement of the thermo stability over the liquid formulation. The physical, chemical and biological deterioration of the subject composition is minimized at both accelerated and real time conditions of storage. This is particularly important for developing countries as this novel composition of lyophilized pegaspargase allows for temperature excursions during transportation and handling.
5. The composition of the lyophilized pegaspargase in the present invention has been co-developed along with the lyophilization process thereby reducing the stress of the lyophilization condition on the product. The high percentage purity of pegaspargase in the novel composition and absence of degraded product, which is often produced as a result of the stress induced by the process of lyophilization on the protein, makes the product less immunogenic (due to the presence of product related impurities—mainly degraded products). Additionally, the present invention does not result in any stress induced aggregation resulting in higher molecular weight species as was observed in case of other prior art products; when analyzed by sodium dodecyl sulfate-polyacrylamide gel electrophoresis (SDS-PAGE). It is to be noted that the liquid composition of prior art product did not show the presence of higher molecular weight species. The novel composition of lyophilized pegaspargase, developed using a novel lyophilized process catered to the composition as detailed out in the present invention, do not show any higher molecular weight species.
6. Based on the optimum amount of excipients and optimized process of lyophilization, the present invention results in a storage stable lyophilized product at optimum cost.

The present invention is illustrated herein by way of examples. The examples provide a description of a composition of the present invention and protection of pegaspargase during lyophilization and storage. The examples are illustration of one embodiment of the present invention and may not in any manner be construed as limiting.

### EXAMPLES

As has been reiterated previously that the lyophilization cycle and the composition needs to be co-developed, since the lyophilization process yielding a storage stable product is dependent on the composition of the formulation which in turn determines the fate of the product post lyophilization. Examples 1 and 2 details the interdependency of the lyophilization process and the composition.

### Example 1: Effect of Excipient

### 1.1 Cryoprotectant

Pegaspargase bulk was buffer exchanged in 50 mM sodium phosphate buffer saline, pH 7.4. The bulk (drug substance) was formulated with various weight percentage (of the composition) of cryo-protectant viz. sucrose and trehalose. 1 ml of the formulated bulk of pegaspargase was filled in pre-sterilized depyrogenated USP type I, 2 mL glass vials (recommended for parenteral) and half stoppered with 13 mm grey bromobutyl coated rubber stopper. The vials were half stoppered and subjected to the lyophilization process.

For the freezing step of the lyophilization process, initial freezing was carried out at −40° C. for 1 hour which was reached a freezing rate of 1.08° C./min, where the vials were held for 3 hours. In the primary drying step, the temperature was brought to −5° C. at a rate of 0.028° C./min under 112 mTorr and was held at that temperature for 6 h. The temperature was further increase to 0° C. at a rate of 0.006° C./min and was maintained at 0° C. for 6 h under 112 mTorr pressure. Lastly, the temperature was further increase to 20° C. at a rate of 0.03° C./min and was maintained at 20° C. for 5 h under 112 mTorr pressure. In the secondary drying step, the pressure was further reduced to 37 mTorr and the temperature was increased to 25° C. at a rate of 0.17° C./min and maintained for 5 h. The total time for the lyophilization process is 76.5 h.

After completion of the lyophilization process the vials were full stoppered by moving the shelf upward. Then the pressure was released by introducing the sterile nitrogen gas in the lyophilization chamber. The lyophilized vials were then sealed with 13 mm flip off seals and subjected to analytical characterization. The cake structure, reconstitution time, clarity post reconstitution, and relative activity (relative to the pre-lyophilized sample) were measured for the lyophilized product. The outcome of the lyophilization process on the various compositions of pegaspargase with varying cryo-protectant is tabulated in Table 1.

TABLE 1
EFFECT OF DIFFERENT CRYO-PROTECTANTS WITH
VARYING PERCENTAGES IN COMPOSITION OF THE
LYOPHILIZED FORMULATION OF PEGASPARGASE

| Sr. | Cryo - | % | Cake | Reconstitution | | Clarity after | Relative |
|---|---|---|---|---|---|---|---|
| No. | protectant | Composition | Appearance | Time | | Reconstitution | % Activity |
| 1 | Sucrose | 35.14% | Poor | 30 | sec | Clear | 89% |
| 2 | | 69.80% | Poor | 30 | sec | Clear | 82% |
| 3 | | 90.24% | Poor | 35 | sec | Clear | 86% |
| 4 | Trehalose | 31.62% | Poor | 27 | sec | Clear | 93% |
| 5 | | 90.24% | Poor | 1:45 | min | Clear | 80% |
| 6 | Sucrose | 55.84% | Poor | 32 | sec | Clear | 95% |
| | Trehalose | 13.96% | | | | | |

The cake structure of all the formulated pegaspargase, in presence of different cryo-protectants with varying percentages of composition of the lyophilized product, were unsatisfactory. Addition of bulking agent was thus required to get a satisfactory outcome.

### 1.2 Bulking Agent

Pegaspargase bulk was buffer exchanged in 50 mM sodium phosphate buffer saline, pH 7.4. The bulk (drug substance) formulated with various weight percentage (of the composition) of bulking agents, viz.—mannitol and glycine. 1 ml of the formulated bulk of pegaspargase was filled in pre-sterilized depyrogenated USP type I, 2 mL glass vials (recommended for parenteral) and half stoppered with 13 mm grey bromobutyl coated rubber stopper. The vials were half stoppered and subjected to the lyophilization process.

For the freezing step of the lyophilization process, initial freezing was carried out at −40° C. for 1 h which was reached a freezing rate of 1.08° C./min, where the vials were held for 3 h. In the primary drying step, the temperature was brought to −35° C. at a rate of 0.028° C./min under 112 mTorr and was held at that temperature for 10 h. The temperature was further increase to 5° C. at a rate of 0.11° C./min and was maintained at 5° C. for 9 h under 112 mTorr pressure. Lastly, the temperature was further increase to 15° C. at a rate of 0.13° C./min and was maintained at 15° C. for 12 h under a reduced pressure of 75 mTorr. In the secondary drying step, the pressure was further reduced to 37 mTorr and the temperature was increased to 25° C. at a rate of 0.33° C./min and maintained for 5 h. The total time for the lyophilization process is 53 h.

After completion of the lyophilization process the vials were full stoppered by moving the shelf upward. Then the pressure was released by introducing the sterile nitrogen gas in the lyophilization chamber. The lyophilized vials were then sealed with 13 mm flip off seals and subjected to analytical characterization. The cake structure, reconstitution time, clarity post reconstitution, and relative activity and purity (relative to the pre-lyophilized sample) were measured for the lyophilized product. The outcome of the lyophilization process on the various compositions of pegaspargase with varying bulking agent is tabulated in Table 2.

TABLE 2
EFFECT OF DIFFERENT BULKING AGENTS WITH VARYING PERCENTAGES
IN COMPOSITION OF THE LYOPHILIZED FORMULATION OF PEGASPARGASE

| Sr. | Bulking | % | Cake | Reconstitution | Clarity after | % | Relative |
|---|---|---|---|---|---|---|---|
| No. | agent | Composition | Appearance | Time | Reconstitution | Activity | % Purity |
| 1 | None | NA | Good | 28 sec | Clear | 69% | 67% |
| 2 | Glycine | 51.54% | Good | 26 sec | Clear | 83% | 98% |
| 3 | Glycine | 51.36% | Good | 52 sec | Clear | 100% | 96% |
| 4 | Mannitol | 35.96% | Poor | 37 sec | Clear | 89% | 97% |
| 5 | Mannitol | 27.44% | Poor | 37 sec | Clear | 83% | 99% |
| | Glycine | 24.01% | | | | | |

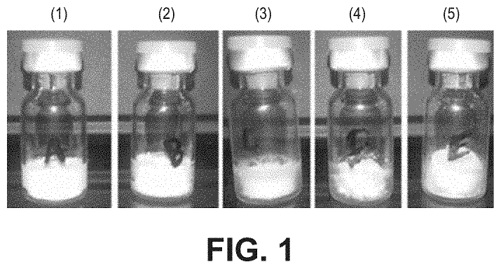

The structure of the cake is shown in FIG. 1. It is clear from the data set that the bulking agent glycine contributes substantially to the cake structure and also retains the activity within permissible limits (600 IU/mL to 900 IU/mL). The cake structure in absence of bulking agent for this lyophilization process is pharmaceutically acceptable, but the activity and the purity of the pegaspargase is very poor. The use of mannitol as a bulking agent, though retains activity and purity does not show a good cake structure for this lyophilization process.

### 1.3 Effect of Salt

Pegaspargase bulk was buffer exchanged in 50 mM sodium phosphate buffer saline, pH 7.4 and formulated with sucrose (cryo/lyo-protectant), different amounts of bulking agent—glycine with varying amount (weight % of the composition) of salt. 2 ml of the formulated bulk was filled in pre-sterilized depyrogenated USP type I, 5 mL glass vials (recommended for parenteral) and half stoppered with 20 mm grey bromobutyl coated rubber stopper. The vials were half stoppered and subjected to the optimized lyophilization process.

The freezing step of the lyophilization process was carried out at −40° C. for 4 h. The freezing temperature was reached at a freezing rate of 1° C./min. In the primary drying step, the temperature was brought to −35° C. at a rate of 0.014° C./min under 112 mTorr and was held at that temperature for 10 h. The temperature was further increase to 5° C. at a rate of 0.06° C./min and was maintained at 5° C. for 9 h under 112 mTorr pressure. The pressure was reduced to 75 mTorr and the temperature was brought to 15° C. at a rate of 0.02° C./min and maintained for 12 h. During the secondary drying cycle, the pressure was further reduced to 37 mTorr and the temperature was increased to 25° C. at a rate of 0.33° C./min and maintained for 5 h.

After completion of the lyophilization process the vials were full stoppered by moving the shelf upward. Then the pressure was released by introducing the sterile nitrogen gas in the lyophilization chamber. The lyophilized vials were then sealed with 20 mm flip off seals. The lyophilized product was reconstituted in 5 mL water for injection and subjected to analytical characterization. The cake structure, reconstitution time, clarity post reconstitution, relative activity (relative to the pre-lyophilization bulk), absolute purity (expressed as percentage as determined by size exclusion high-performance liquid chromatography (SE-HPLC)) and osmolality were measured for the lyophilized product. The outcome of the lyophilization process on the various compositions of pegaspargase is tabulated in Table 3.

TABLE 3
THE EFFECT OF NACL AND GLYCINE ON THE LYOPHILIZATION OF PEGASPARGASE

| | % | | | Reconstitution | Clarity | | | |
|---|---|---|---|---|---|---|---|---|
| Sr. | Composition | | Cake | Time | after | % | % | Osmolality |
| No. | NaCl | Glycine | Appearance | (sec) | Reconstitution | Activity | Purity | (mOsm/Kg) |
| 1 | 0.00% | 48.44% | Good | 40 | Clear | 90.0% | 98.0% | 427 |
| 2 | 0.00% | 46.45% | Good | 39 | Clear | 90.7% | 97.7% | 393 |
| 3 | 0.00% | 44.29% | Good | 42 | Clear | 80.8% | n.d. | 370 |
| 4 | 0.44% | 48.23% | Good | 44 | Clear | 92.6% | 97.6% | 427 |
| 5 | 0.45% | 46.24% | Good | 35 | Clear | 96.8% | 98.2% | 395 |
| 6 | 0.47% | 44.08% | Good | 30 | Clear | 93.9% | 97.9% | 385 |
| 7 | 1.08% | 47.92% | Good | 35 | Clear | 85.3% | 97.6% | 433 |
| 8 | 1.12% | 45.93% | Good | 33 | Clear | 87.8% | 97.6% | 403 |
| 9 | 1.16% | 43.78% | Good | 32 | Clear | 61.9% | n.d. | 384 |

It is evident that the optimized lyophilization process can be used for the composition with low and no salt concentration without altering the critical product attributes.

### Example 2: Effect of Different Lyophilization Cycle on the Same Composition

Pegaspargase bulk was buffer exchanged in 50 mM sodium phosphate buffer saline, pH 7.4. The bulk (drug substance) was formulated with sucrose (cryo/lyo-protectant at 34.3% of the composition), and glycine (bulking agent at 51.5% of the composition). 1 ml of the formulated bulk was filled in pre-sterilized depyrogenated USP type I, 2 mL glass vials (recommended for parenteral) and half stoppered with 13 mm grey bromobutyl coated rubber stopper. The vials were half stoppered and subjected to various lyophilization process.

The freezing step of the various lyophilization process was carried out for various duration and temperature. In some cases, a single step freezing was carried out while in others multi-step freezing was carried out. In one cycle the initial freezing was carried out at −15° C. for 2 h which was reached a freezing rate of 1.16° C./min. This was followed with a further decrease of temperature to −25° C., achieved at the freezing rate of 0.33° C./min where the vials were held for 3 h. Lastly the temperature was brought down to −40° C., achieved at the freezing rate of 0.5° C./min where the vials were held for 2 h. The total freezing step duration was 8.5 h. In another cycle the freezing step of the lyophilization process was carried out at −40° C. for 3 h. The freezing temperature was reached at a freezing rate of 1° C./min. The total freezing step duration was 4 h. In another cycle the freezing step of the lyophilization process was carried out at −40° C. for 6 h. The freezing temperature was reached at a freezing rate of 0.5° C./min. The total freezing step duration was 8 h. Yet, in another cycle the freezing step of the lyophilization process was carried out at −40° C. for 4 h. The freezing temperature was reached at a freezing rate of 1° C./min. The total freezing step duration was 5 h.

The primary drying step of the lyophilization cycle was also varied with respect to temperature, pressure and time. In one cycle, for the primary drying step, the temperature was brought to −5° C. at a rate of 0.028° C./min under 112 mTorr and was held at that temperature for 6 h. The temperature was further increase to 0° C. at a rate of 0.006° C./min and was maintained at 0° C. for 6 h under 112 mTorr pressure. Lastly, the temperature was further increase to 20° C. at a rate of 0.03° C./min and was maintained at 20° C. for 5 h under 112 mTorr pressure. The total primary drying step duration was 61.5 h. In another cycle for the primary drying step, the temperature was brought to −5° C. at a rate of 0.15° C./min under 112 mTorr and was held at that temperature for 14 h. The temperature was further increase to 5° C. at a rate of 0.06° C./min and was maintained at 5° C. for 9 h under 112 mTorr pressure. Lastly, pressure was reduced to 75 mTorr and the temperature was brought to 15° C. at a rate of 0.067° C./min and maintained for 6 h. The total primary drying step duration was 38.5 h. In another cycle for the primary drying step, the temperature was brought to −35° C. at a rate of 0.027° C./min under 112 mTorr and was held at that temperature for 10 h. The temperature was further increase to 5° C. at a rate of 0.11° C./min and was maintained at 5° C. for 9 h under 112 mTorr pressure. Lastly, pressure was reduced to 75 mTorr and the temperature was brought to 15° C. at a rate of 0.067° C./min and maintained for 12 h. The total primary drying step duration was 42.5 h. In another cycle for the primary drying step, the temperature was brought to −35° C. at a rate of 0.027° C./min under 112 mTorr and was held at that temperature for 10 h. The temperature was further increase to 5° C. at a rate of 0.11° C./min and was maintained at 5° C. for 9 h under 112 mTorr pressure. The temperature was further increase to 10° C. at a rate of 0.014° C./min and was maintained at 10° C. for 24 h under 112 mTorr pressure. Lastly, pressure was reduced to 75 mTorr and the temperature was brought to 15° C. at a rate of 0.033° C./min and maintained for 12 h. The total primary drying step duration was 72.5 h. Yet, in another cycle for the primary drying step, the temperature was brought to −35° C. at a rate of 0.027° C./min under 112 mTorr and was held at that temperature for 6 h. The temperature was further increase to 15° C. at a rate of 0.138° C./min and was maintained at 15° C. for 9 h under 112 mTorr pressure. Lastly, pressure was reduced to 75 mTorr over 5 h at 15° C. and maintained for 12 h. The total primary drying step duration was 38.5 h.

The secondary drying step of the lyophilization cycle was also varied with respect to temperature, pressure and time. In one cycle, for the secondary drying step the pressure was further reduced to 37 mTorr and the temperature was increased to 25° C. at a rate of 0.16° C./min and maintained for 5 h. The total secondary drying step duration was 5.5 h. In one cycle, for the secondary drying step the pressure was further reduced to 37 mTorr and the temperature was increased to 25° C. at a rate of 0.33° C./min and maintained for 5 h. The total secondary drying step duration was 5.5 h. Yet, in one cycle, for the secondary drying step the pressure was further reduced to 37 mTorr and the temperature was increased to 25° C. at a rate of 0.33° C./min and maintained for 9 h. The total secondary drying step duration was 9.5 h.

The completion time for the lyophilization process varied from 48 h to 83.5 h.

After completion of the lyophilization process the vials were full stoppered by moving the shelf upward. Then the pressure was released by introducing the sterile nitrogen gas in the lyophilization chamber. The lyophilized vials were then sealed with 13 mm flip off seals and subjected to analytical characterization. The cake structure, reconstitution time, clarity post reconstitution, relative activity (relative to the pre lyophilization bulk), relative purity (relative to the pre lyophilization bulk as determined by size exclusion high-performance liquid chromatography (SE-HPLC)) and osmolality were measured for the lyophilized product. The lyophilized product had good to satisfactory cake formation, acceptable reconstitution time (less than 2 minutes) and the reconstituted sample was clear and colorless. However, the relative activity and purity varied considerably as shown in Table 4. This outcome is expected due to different stress conditions imparted on the same composition due to change in the lyophilization process.

TABLE 4
RELATIVE PURITY AND ACTIVITY OF
THE SAME FORMULATION WITH VARYING
LYOPHILIZATION CONDITIONS

| | Time (hr.) | | | | Relative | Relative |
|---|---|---|---|---|---|---|
| Lyophilization | Freez- | Primary | Secondary | | % | % |
| Cycles | ing | Drying | Drying | Total | Activity | Purity |
| 1 | 8.5 | 61.5 | 5.5 | 75.5 | 84% | 83% |
| 2 | 4.0 | 61.5 | 5.5 | 71.0 | 98% | 91% |
| 3 | 4.0 | 38.5 | 5.5 | 48.0 | 91% | 93% |
| 4 | 8.0 | 38.5 | 5.5 | 52.0 | 87% | 91% |
| 5 | 5.0 | 42.5 | 5.5 | 53.0 | 83% | 98% |
| 6 | 5.0 | 61.5 | 5.5 | 72.0 | 87% | 101% |
| 7 | 5.0 | 38.5 | 9.5 | 53.0 | 85% | 95% |

### Example 3: An Illustrative Example of the Composition of the Present Invention—Low Salt

Pegaspargase bulk was buffer exchanged in 50 mM sodium phosphate buffer saline, pH 7.4. The required bulk was formulated with sucrose as the cryo/lyo-protectant and glycine as the bulking agent and finally diluted to achieve 1875 IU/mL. The final formulation comprised of pegaspargase, sucrose, glycine, sodium phosphate monobasic, sodium phosphate dibasic and sodium chloride at 13.67% to 7.04%, 39.60% to 36.78%, 47.52% to 44.13%, 0.95% to 0.88%, 4.42% to 4.10% and 0.47% to 0.43% of the composition respectively. 2 ml of the formulated bulk was filled in pre-sterilized depyrogenated USP type I, 5 mL glass vials (recommended for parenteral) and half stoppered with 20 mm grey bromobutyl coated rubber stopper. The vials were half stoppered and subjected to the optimized lyophilization process.

The freezing step of the lyophilization process was carried out at −40° C. for 4 h. The freezing temperature was reached at a freezing rate of 1° C./min. In the primary drying cycle, the temperature was brought to −35° C. at a rate of 0.014° C./min under 112 mTorr and was held at that temperature for 10 h. The temperature was further increase to 5° C. at a rate of 0.06° C./min and was maintained at 5° C. for 9 h under 112 mTorr pressure. The pressure was reduced to 75 mTorr and the temperature was brought to 15° C. at a rate of 0.02° C./min and maintained for 12 h. During the secondary drying cycle, the pressure was further reduced to 37 mTorr and the temperature was increased to 25° C. at a rate of 0.33° C./min and maintained for 5 h. The total time for the lyophilization process is 66.5 h.

After completion of the lyophilization process the vials were full stoppered by moving the shelf upward. Then the pressure was released by introducing the sterile nitrogen gas in the lyophilization chamber. The lyophilized vials were then sealed with 20 mm flip off seals. The lyophilized product was reconstituted in 5 mL water for injection and subjected to analytical characterization (Table 5).

TABLE 5
ANALYTICAL CHARACTERIZATION OF LYOPHILIZED PEGASPARGASE

| Test | Acceptance Criteria | Lyophilized Product |
|---|---|---|
| Appearance | Pre-reconstitution - White to off white cake | Complies |
| | Post-reconstitution - Colorless solution | |
| Reconstitution Time (sec) | NMT 180 sec | 68 |
| Clarity | Clear, no visible particles | Complies |
| Fill Volume | To deliver 5.0 mL (USP) | 5.0 mL |
| pH | 7.0-7.4 | 7.08 |
| Potency (Activity) | 600-900 IU/mL | 680 |
| Specific Activity | ≥85 IU/mg protein | 128 |
| Purity by SEC | | 94.94% |
| Relative Purity | ≥85% active components, ≤8% | 99.11% |
| | aggregates | |
| Protein Concentration | 4.8-8.5 mg/mL | 5.33 |
| Moisture Content | NMT 5% | 3.5% |
| Osmolality (mOsm/kg) | 270-600 mOsm/kg | 409 |

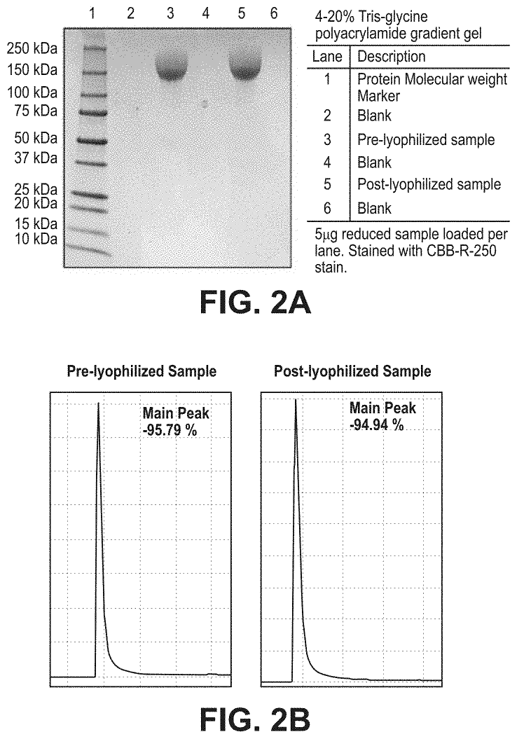

FIG. 2 demonstrates the integrity and purity of the pre and the post lyophilized pegaspargase as determined by sodium dodecyl sulfate-polyacrylamide gel electrophoresis (SDS-PAGE) and size exclusion high-performance liquid chromatography (SE-HPLC) respectively.

The process robustness of the lyophilization cycle as well as the composition was verified over multiple batches. The cake structure, reconstitution time, post reconstitution clarity, absolute activity, relative activity (relative to the pre lyophilization bulk), absolute purity (expressed as percentage as determined by size exclusion high-performance liquid chromatography (SE-HPLC)), relative purity (relative to the pre lyophilization bulk), osmolality and moisture content were measured for the lyophilized product. The product characteristics complies with the acceptance criteria as shown in Table 6 for three representative batches.

TABLE 6
ANALYTICAL CHARACTERIZATION OF THREE LOTS OF LYOPHILIZED PEGASPARGASE

| Test | Acceptance Criteria | Lot 1 | Lot 2 | Lot 3 |
|---|---|---|---|---|
| Appearance | Pre-reconstitution - White to off white cake | Complies | Complies | Complies |
| | Post-reconstitution - Colorless solution | Complies | Complies | Complies |
| Reconstitution Time (sec) | NMT 180 sec | 59 | 59 | 66 |
| Clarity | Clear, no visible particles | Complies | Complies | Complies |
| Potency (Activity) | 600-900 IU/mL | 815 | 869 | 727 |
| Relative Activity | NLT 85% | 103.70% | 101.60% | 97.60% |
| Purity by SEC | ≥85% active components, ≤8% | 97.55% | 99.73% | 99.78% |
| Relative Purity | aggregates | 100.40% | 100.00% | 100.00% |
| Moisture Content | NMT 5% | 2.28% | 2.78% | 2.35% |
| Osmolality (mOsm/kg) | 270-600 mOsm/kg | 390 | 398 | 401 |

The lyophilized product was subjected to long term stability study at 5° C.±3° C. for 24 months and accelerated stability study at 25° C.±2° C./60%±5% relative humidity for 6 months as per ICH quality guidelines (Q1A). The outcome of the lyophilization process on the product pegaspargase at various time point at ambient temperature (5° C.±3° C.) and at 25° C.±2° C./60%±5% relative humidity is tabulated in and Error! Reference source not found. and

TABLE 7
LONG TERM STABILITY STUDY OF LYOPHILIZED PEGASPARGASE AT 5° C. ± 3° C. FOR 24 MONTHS.

| | Acceptance | 0 | 15 | 1 | 3 | 6 | 9 | 12 | 18 | 24 |
|---|---|---|---|---|---|---|---|---|---|---|
| Test | Criteria | day | day | Month | Months | Months | Months | Months | Months | Months |
| Appearance | Pre-reconstitution - | Complies | | | | | | | | |
| | White to off white cake | | | | | | | | | |
| | Post-reconstitution - | | | | | | | | | |
| | Colorless solution | | | | | | | | | |
| Reconstitution | NMT 180 sec | 68 | 58 | 58 | 52 | 58 | 59 | 59 | 57 | 54 |
| Time (sec) | | | | | | | | | | |
| Clarity | Clear, no visible | Complies | | | | | | | | |
| | particles | | | | | | | | | |
| Fill Volume | To deliver 5.0 mL | 5.0 mL | 5.0 mL | 5.0 mL | 5.0 mL | 5.0 mL | 5.0 mL | 5.0 mL | 5.0 mL | 5.0 mL |
| | (USP) | | | | | | | | | |
| pH | 7.0-7.4 | 7.08 | 7.09 | 7.11 | 7.07 | 7.08 | 7.2 | 7.19 | 7.17 | 7.2 |
| Potency | 600-900 IU/mL | 680 | 687 | 679 | 673 | 661 | 650 | 656 | 675 | 642 |
| (Activity) | | | | | | | | | | |
| Specific | ≥85 IU/mg protein | 128 | 128 | 126 | 130 | 127 | 115 | 120 | 121 | 123 |
| Activity | | | | | | | | | | |
| Purity by SEC | ≥85% active | 94.94% | 95.96% | 95.86% | 95.82% | 95.77% | 95.70% | 95.79% | 95.40% | 93.05% |
| | components, ≤8% | | | | | | | | | |
| Relative Purity | aggregates | 99.11% | 100.18% | 100.07% | 100.03% | 99.98% | 99.91% | 100.00% | 99.59% | 97.14% |
| Protein | 4.8-8.5 mg/mL | 5.33 | 5.35 | 5.39 | 5.18 | 5.2 | 5.66 | 5.48 | 5.58 | 5.24 |
| Concentration | | | | | | | | | | |
| Osmolality | 270-600 mOsm/kg | 409 | 419 | 412 | 411 | 413 | 406 | 411 | 408 | 413 |
| (mOsm/kg) | | | | | | | | | | |

Table 8 respectively.

| Appearance | Pre-reconstitution - | Complies | | | | | | | | |
|---|---|---|---|---|---|---|---|---|---|---|
| | White to off white cake | | | | | | | | | |
| | Post-reconstitution - | | | | | | | | | |
| | Colorless solution | | | | | | | | | |
| Reconstitution | NMT 180 sec | 68 | 58 | 58 | 52 | 58 | 59 | 59 | 57 | 54 |
| Time (sec) | | | | | | | | | | |
| Clarity | Clear, no visible | Complies | | | | | | | | |
| | particles | | | | | | | | | |
| Fill Volume | To deliver 5.0 mL | 5.0 mL | 5.0 mL | 5.0 mL | 5.0 mL | 5.0 mL | 5.0 mL | 5.0 mL | 5.0 mL | 5.0 mL |
| | (USP) | | | | | | | | | |
| pH | 7.0-7.4 | 7.08 | 7.09 | 7.11 | 7.07 | 7.08 | 7.2 | 7.19 | 7.17 | 7.2 |
| Potency | 600-900 IU/mL | 680 | 687 | 679 | 673 | 661 | 650 | 656 | 675 | 642 |
| (Activity) | | | | | | | | | | |
| Specific | ≥85 IU/mg protein | 128 | 128 | 126 | 130 | 127 | 115 | 120 | 121 | 123 |
| Activity | | | | | | | | | | |
| Purity by SEC | ≥85% active | 94.94% | 95.96% | 95.86% | 95.82% | 95.77% | 95.70% | 95.79% | 95.40% | 93.05% |
| | components, ≤8% | | | | | | | | | |
| Relative Purity | aggregates | 99.11% | 100.18% | 100.07% | 100.03% | 99.98% | 99.91% | 100.00% | 99.59% | 97.14% |
| Protein | 4.8-8.5 mg/mL | 5.33 | 5.35 | 5.39 | 5.18 | 5.2 | 5.66 | 5.48 | 5.58 | 5.24 |
| Concentration | | | | | | | | | | |
| Osmolality | 270-600 mOsm/kg | 409 | 419 | 412 | 411 | 413 | 406 | 411 | 408 | 413 |
| (mOsm/kg) | | | | | | | | | | |

TABLE 8
ACCELERATED STABILITY STUDY OF LYOPHILIZED PEGASPARGASE AT 25° C. ±
3° C./60% ± 5% RELATIVE HUMIDITY FOR 6 MONTHS.

| | Acceptance | 0 | 15 | 1 | 3 | 6 |
|---|---|---|---|---|---|---|
| Test | Criteria | day | day | Months | Months | Months |
| Appearance | Pre-reconstitution - | Complies | | | | |
| | White to off white cake | | | | | |
| | Post-reconstitution - | | | | | |
| | Colorless solution | | | | | |
| Reconstitution | NMT 180 sec | 68 | 64 | 63 | 56 | 62 |
| Time (sec) | | | | | | |
| Clarity | Clear, no visible | Complies | | | | |
| | particles | | | | | |
| Fill Volume | To deliver 5.0 mL | 5.0 mL | 5.0 mL | 5.0 mL | 5.0 mL | 5.0 mL |
| | (USP) | | | | | |
| pH | 7.0-7.4 | 7.08 | 7.1 | 7.1 | 7.06 | 7.05 |
| Potency | 600-900 IU/mL | 680 | 679 | 681 | 657 | 622 |
| (Activity) | | | | | | |
| Specific | ≥85 IU/mg protein | 128 | 129 | 132 | 123 | 119 |
| Activity | | | | | | |
| Purity by SEC | ≥85% active | 94.94% | 95.53% | 95.00% | 95.09% | 94.06% |
| | components, ≤8% | | | | | |
| Relative Purity | aggregates | 99.11% | 99.73% | 99.18% | 99.27% | 98.19% |
| Protein | 4.8-8.5 mg/mL | 5.33 | 5.28 | 5.14 | 5.33 | 5.24 |
| Concentration | | | | | | |
| Osmolality | 270-600 mOsm/kg | 409 | 412 | 414 | 411 | 410 |
| (mOsm/kg) | | | | | | |

The product characteristics complies with the acceptance criteria for the entire duration showing that the product is stable at 5° C.±3° C. for 24 months and at 25° C.±2° C./60%±5% relative humidity for 6 months. Additionally, the stability of the lyophilized product was evaluated at elevated temperature for which the product was subjected to long term stability study at 30° C.±2° C./65%±5% relative humidity for 18 months and accelerated stability study at 37° C.±2° C./75%±5% relative humidity for 3 months. The outcome of the lyophilization process on the product pegaspargase at various time point at 30° C.±2° C./65%±5% relative humidity and at 37° C.±2° C./75%±5% relative humidity is tabulated in Error! Reference source not found. and Table 10 respectively. The product characteristics complies with the acceptance criteria for the entire duration showing that the product is stable at 30° C.±2° C./65%±5% relative humidity for 18 months and at 37° C.±2° C./75%±5% relative humidity relative humidity for 3 months.

TABLE 9
LONG TERM STABILITY STUDY OF LYOPHILIZED PEGASPARGASE AT 30° C. ±
2° C./65% ± 5% RELATIVE HUMIDITY FOR 18 MONTHS

| | Acceptance | 0 | 15 | 1 | 3 | 6 | 9 | 12 | 18 |
|---|---|---|---|---|---|---|---|---|---|
| Test | Criteria | day | day | Months | Months | Months | Months | Months | Months |
| Appearance | Pre-reconstitution - | Complies | | | | | | | |
| | White to off white cake | | | | | | | | |
| | Post-reconstitution - | | | | | | | | |
| | Colorless solution | | | | | | | | |
| Reconstitution | NMT 180 sec | 68 | 57 | 59 | 58 | 61 | 58 | 62 | 61 |
| Time (sec) | | | | | | | | | |
| Clarity | Clear, no visible | Complies | | | | | | | |
| | particles | | | | | | | | |
| Fill Volume | To deliver 5.0 | 5.0 mL | 5.0 mL | 5.0 mL | 5.0 mL | 5.0 mL | 5.0 mL | 5.0 mL | 5.0 mL |
| | mL (USP) | | | | | | | | |
| pH | 7.0-7.4 | 7.08 | 7.08 | 7.09 | 7.07 | 7.05 | 7.27 | 7.18 | 7.24 |
| Potency | 600-900 IU/mL | 680 | 668 | 687 | 652 | 611 | 617 | 629 | 601 |
| (Activity) | | | | | | | | | |
| Specific | ≥85 IU/mg | 128 | 131 | 138 | 128 | 119 | 116 | 112 | 113 |
| Activity | protein | | | | | | | | |
| Purity by SEC | ≥85% active | 94.94% | 95.31% | 95.00% | 94.11% | 91.11% | 90.10% | 91.00% | 87.06% |
| | components, ≤8% | | | | | | | | |
| Relative Purity | aggregates | 99.11% | 99.50% | 99.18% | 98.25% | 95.11% | 94.06% | 95.00% | 90.89% |
| Protein | 4.8-8.5 mg/mL | 5.33 | 5.11 | 4.98 | 5.08 | 5.15 | 5.33 | 5.61 | 5.3 |
| Concentration | | | | | | | | | |
| Osmolality | 270-600 mOsm/kg | 409 | 411 | 415 | 406 | 409 | 396 | 409 | 412 |
| (mOsm/kg) | | | | | | | | | |

TABLE 10
ACCELERATED STABILITY STUDY OF LYOPHILIZED PEGASPARGASE AT 37° C. ±
2 ° C./75% ± 5% RELATIVE HUMIDITY FOR 3 MONTHS

| Test | Acceptance Criteria | 0 day | 15 day | 1 Months | 3 Months |
|---|---|---|---|---|---|
| Appearance | Pre-reconstitution - White to off white cake | Complies | | | |
| | Post-reconstitution - Colorless solution | | | | |
| Reconstitution Time (sec) | NMT 180 sec | 68 | 56 | 54 | 59 |
| Clarity | Clear, no visible particles | Complies | | | |
| Fill Volume | To deliver 5.0 mL (USP) | 5.0 mL | 5.0 mL | 5.0 mL | 5.0 mL |
| pH | 7.0-7.4 | ‘ 7.08 | 7.09 | 7.09 | 7.1 |
| Potency (Activity) | 600-900 IU/mL | 680 | 656 | 699 | 631 |
| Specific Activity | ≥85 IU/mg protein | 128 | 125 | 130 | 125 |
| Purity by SEC | ≥85% active components, | 94.94% | 91.16% | 91.15% | 90.26% |
| Relative Purity | ≤8% aggregates | 99.11% | 95.17% | 95.16% | 94.23% |
| Protein Concentration | 4.8-8.5 mg/mL | 5.33 | 5.24 | 5.37 | 5.05 |
| Osmolality (mOsm/kg) | 270-600 mOsm/kg | 409 | 411 | 419 | 410 |

### Example 4: An Illustrative Example of the Composition of the Present Invention—No Salt

Pegaspargase bulk was buffer exchanged in 50 mM sodium phosphate buffer, pH 7.4. The required concentrated bulk was formulated with sucrose as the cryo/lyo-protectant and glycine as the bulking agent and finally diluted to achieve 1875 IU/mL. The final formulation comprised of pegaspargase, sucrose, glycine, sodium phosphate monobasic and sodium phosphate dibasic at 12.57% to 9.60%, 38.71% to 37.43%, 46.45% to 44.92%, 0.93% to 0.90% and 4.32% to 4.18% of the composition respectively. 2 ml of the formulated bulk was filled in pre-sterilized depyrogenated USP type I, 5 mL glass vials (recommended for parenteral) and half stoppered with 20 mm grey bromobutyl coated rubber stopper. The vials were half stoppered and subjected to the optimized lyophilization process.

The freezing step of the lyophilization process was carried out at −40° C. for 4 h. The freezing temperature was reached at a freezing rate of 1° C./min. In the primary drying cycle the temperature was brought to −35° C. at a rate of 0.014° C./min under 112 mTorr and was held at that temperature for 10 h. The temperature was further increase to 5° C. at a rate of 0.06° C./min and was maintained at 5° C. for 9 h under 112 mTorr pressure. The pressure was reduced to 75 mTorr and the temperature was brought to 15° C. at a rate of 0.02° C./min and maintained for 12 h. During the secondary drying cycle, the pressure was further reduced to 37 mTorr and the temperature was increased to 25° C. at a rate of 0.33° C./min and maintained for 5 h. The total time for the lyophilization process is 66.5 h.

After completion of the lyophilization process the vials were full stoppered by moving the shelf upward. Then the pressure was released by introducing the sterile nitrogen gas in the lyophilization chamber. The lyophilized vials were then sealed with 20 mm flip off seals. The lyophilized product was reconstituted in 5 mL water for injection and subjected to analytical characterization (Table 11).

TABLE 11
ANALYTICAL CHARACTERIZATION OF LYOPHILIZED
PEGASPARGASE AS NO SALT COMPOSITION

| Test | Acceptance Criteria | Lyophilized Product |
|---|---|---|
| Appearance | Pre-reconstitution - White to off white cake | Complies |
| | Post-reconstitution - Colorless solution | |
| Reconstitution Time (sec) | NMT 180 sec | 49 |
| Clarity | Clear, no visible particles | Complies |
| Fill Volume | To deliver 5.0 mL (USP) | 5.0 mL |
| Potency (Activity) | 600-900 IU/mL | 803 |
| Specific Activity | ≥85 IU/mg protein | 144 |
| Purity by SEC | ≥85% active components, ≤8% | 99.40% |
| Relative Purity | aggregates | 100% |
| Protein Concentration | 4.8-8.5 mg/mL | 5.56 |
| Moisture Content | NMT 5% | 2.47% |
| Osmolality (mOsm/kg) | 270-600 mOsm/kg | 434 |

The process robustness of the lyophilization cycle as well as the composition was verified over multiple batches. The cake structure, reconstitution time, post reconstitution clarity, absolute activity, relative activity (relative to the pre-lyophilization bulk), absolute purity (expressed as percentage as determined by size exclusion high-performance liquid chromatography (SE-HPLC)), relative purity (relative to the pre-lyophilization bulk), osmolality and moisture content were measured for the lyophilized product. The product characteristics complies with the acceptance criteria as shown in Table 12 for three representative batches.

TABLE 12
ANALYTICAL CHARACTERIZATION OF THREE LOTS OF LYOPHILIZED
PEGASPARGASE AS NO SALT COMPOSITION

| Test | Acceptance Criteria | Lot 1 | Lot 2 | Lot 3 |
|---|---|---|---|---|
| Appearance | Pre-reconstitution - White to off white cake | Complies | Complies | Complies |
| | Post-reconstitution - Colorless solution | | | |
| Reconstitution Time (sec) | NMT 180 sec | 49 | 83 | 49 |
| Clarity | Clear, no visible particles | Complies | Complies | Complies |
| Potency (Activity) | 600-900 IU/mL | 831 | 849 | 803 |
| Relative Activity | NLT 85% | 89% | 107% | 101% |
| Purity by SEC | ≥85% active components, ≤8% | 99% | 99% | 99% |
| Relative Purity | aggregates | 99% | 100% | 100% |
| Moisture Content | NMT 5% | 2.20% | 2.37% | 2.47% |
| Osmolality (mOsm/kg) | 270-600 mOsm/kg | 420 | 444 | 434 |

The lyophilized product without salt was subjected to long term stability study at 5° C.±3° C., at 25° C.±2° C./60%±5%, at 30° C.±2° C./65%±5% relative humidity and at 37° C.±2° C./75%±5% relative humidity and the data is presented in Table 13 for one month and in Table 14 for three months.

TABLE 13
STABILITY OF AN EMBODIMENT OF THE COMPOSITION
OF THE PRESENT INVENTION AT 1 MONTH

| | Acceptance | | 1 Month (Storage temperature) | | | |
|---|---|---|---|---|---|---|
| Tests | Criteria | 0 day | 5° C. | 25° C. | 30° C. | |
| Appearance | White to off white | White to off white | White to off white intact cake, tightly attached to vial. | | | |
| | intact cake, tightly | intact cake, tightly | | | | |
| | attached to vial. | attached to vial. | | | | |
| Reconstitution | ≤180 sec | 128 | 107 | 93 | 108 | 87 |
| Time (s) | | | | | | |
| Clarity after | Clear Solution no | Clear Solution no | Clear Solution no visible | | | |
| reconstitution | visible particle | visible particle | particle should be observed | | | |
| | should be observed | should be observed | | | | |
| pH | 7.1 to 7.5 | 7.3 | 7.23 | 7.24 | 7.23 | 7.24 |
| Protein content | NLT 5 mg/mL | 5.53 | 5.8 | 6.22 | 6.06 | 5.91 |
| (mg/mL) | | | | | | |
| Assay (IU/mL) | 600-900 IU/mL | 753 | 722 | 753 | 729 | 748 |
| Specific activity | NLT 85 IU/mg | 136 | 124 | 121 | 120 | 127 |
| (IU/mg) | | | | | | |
| Osmolality | NMT 460 | 430 | 434 | 436 | 439 | 445 |
| (mOsm/kg) | mOsm/kg | | | | | |
| Purity by SEC (%) | NLT 95% | 99.63 | 99.95 | 99.99 | 97.76 | 96.83 |
| PMT/Total No. of | NMT 6000 | ≥10 pm size:−232 | 45 | 30 | 30 | 40 |
| particles per | NMT 600 | ≥25 pm size:−020 | 05 | 03 | 05 | 03 |
| Container | | | | | | |

| | |
|---|---|
| SDS-PAGE | Acceptable |

TABLE 14
STABILITY OF AN EMBODIMENT OF THE COMPOSITION
OF THE PRESENT INVENTION AT 3 MONTH

| | | | 3 Month (Storage | | | |
|---|---|---|---|---|---|---|
| | Acceptance | | temperature) | | | |
| Tests | Criteria | 0 day | 5° C. | 25° C. | 30° C. | 37° C. |
| Appearance | White to off white | White to off white | White to off white intact cake, | | | |
| | intact cake, tightly | intact cake, tightly | tightly attached to vial. | | | |
| | attached to vial. | attached to vial. | | | | |
| Reconstitution | ≤180 sec | 128 | 65 | 80 | 50 | 90 |
| Time (s) | | | | | | |
| Clarity after | Clear Solution no | Clear Solution no | Clear Solution no visible | | | |
| reconstitution | visible particle | visible particle | particle should be observed | | | |
| | should be observed | should be observed | | | | |
| pH | 7.1 to 7.5 | 7.3 | 7.29 | 7.29 | 7.29 | 7.28 |
| Protein content | NLT 5 mg/mL | 5.53 | 6.21 | 6.12 | 5.56 | 5.66 |
| (mg/mL) | | | | | | |
| Assay (IU/mL) | 600-900 IU/mL | 753 | 748 | 719 | 708 | 763 |
| Specific activity | NLT 85 IU/mg | 136 | 120 | 116 | 127 | 135 |
| (IU/mg) | | | | | | |
| Osmolality | NMT 460 | 430 | 439 | 434 | 432 | 440 |
| (mOsm/kg) | mOsm/kg | | | | | |
| Purity by SEC | NLT 95% | 99.63 | 99.39 | 99.31 | 97.67 | 95.87 |
| (%) | | | | | | |
| PMT/Total No. of | NMT 6000 | ≥10 pm size:−232 | 23 | 23 | 30 | 73 |
| particles per | NMT 6000 | ≥25 pm size:−020 | 05 | 03 | 05 | 03 |
| Container | | | | | | |
| SDS-PAGE | | Acceptable | Acceptable | | | |

### Example 5: Comparison of the Composition of the Present Invention with the Prior Art

### 5.1 Liquid Composition of Prior Art Vs Solid Composition of the Present Invention

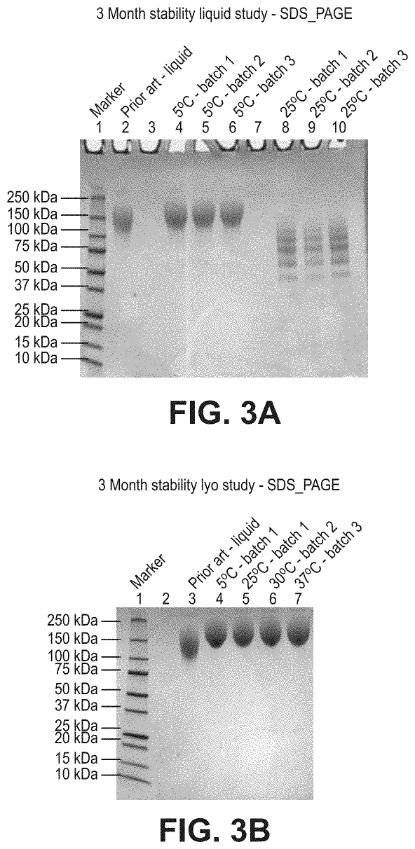

Pegaspargase reported in prior art is generally presented as liquid composition and not storage stable at longer duration and at higher temperature. The present invention discloses a storage stable lyophilized composition at various temperatures. It is mentioned in the prior art that pegaspargase in not stable at longer duration. The current invention overcome this limitation and provide a storage stable composition. The prior art product degrades substantially within 3 months at 25° C.±2° C./60%±5% relative humidity. As shown in FIG. 3, the degradation of the liquid formulation is evident by the presence of multiple bands at 25° C.±2° C./60%±5% relative humidity for the liquid composition as analyzed by SDS-PAGE (FIG. 3 (A)). This invention produces a storage stable formulation which is stable at 25° C.±2° C./60%±5% relative humidity, 30° C.±2° C./65%±5% relative humidity and at 37° C. 2° C./75%±5% relative humidity. The stability of the present composition is evident from the presence of a single diffused band at appropriate molecular weight as shown in FIG. 3 (B)

### 5.2 Solid Composition of Prior Art Vs Solid Composition of the Present Invention

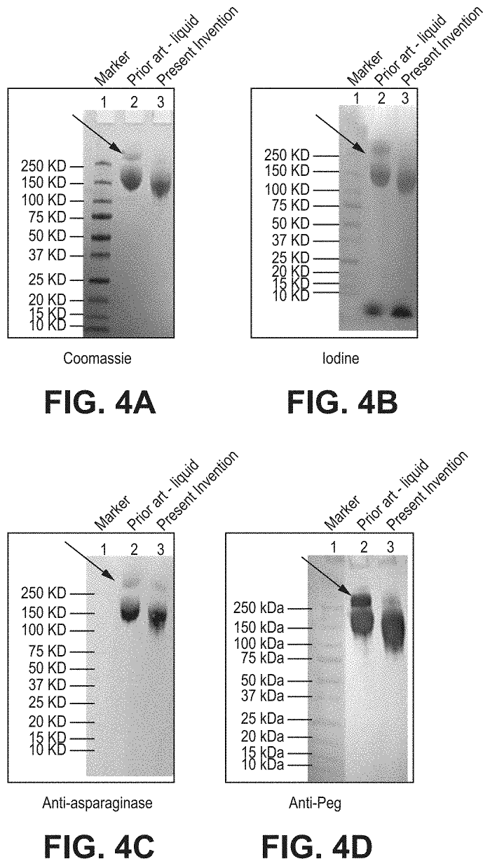

Prior art discloses a storage stable composition but have limitation in terms of high molecular weight aggregates in lyophilized product. The current disclosure uses an inventive process and improved composition, and does not have any further aggregation/high molecular impurities (see FIG. 4). FIG. 4 shows SDS-PAGE analysis of prior art lyophilized composition with the current invention. The SDS-PAGE gels confirm the presence of high molecular weight impurity as evident from the Coomassie staining for protein (FIG. 4(A)) and Iodine staining (FIG. 4(B)) for PEG. Additionally, Western Blot analysis with anti-asparaginase antibody (FIG. 4(C)) and Anti-PEG antibody (FIG. 4(B)) confirms the presence of product related impurities. This further highlights the synergistic relationship of the lyophilization process with the composition of the formulation. It is to be noted that the liquid composition of prior art product did not show the presence of higher molecular weight species.

1. An optimum storage stable lyophilized composition comprising pegaspargase, a cryoprotectant, a bulking agent, a buffer, optionally a pharmaceutically acceptable excipient.

2. The composition as claimed in claim 1, wherein the pegaspargase is a pegylated asparaginase comprising a polyalkylene oxide group covalently linked by a linker to asparaginase; wherein the polyalkylene oxide is mono-methoxy polyethylene glycol (mPEG) and is covalently linked by a succinate linker via an amide bond to one or more primary amine groups of an L-asparaginase through conjugation; wherein conjugation of mPEG and L-asparaginase results in covalent attachment of 1-12 mPEGs per monomer of L-asparaginase.

3. The composition as claimed in claim 2, wherein L-Asparaginase is from a bacterial source selected from the group consisting of E. coli and Erwinia chrysanthemi or obtained through genetically engineered E. coli through recombinant technology.

4. The composition as claimed in claim 1, wherein an amount of pegaspargase in the composition is 2-32 of the composition.

5. The composition as claimed in claim 1, wherein the cryoprotectant is selected from the group consisting of sugar, polyol, polymer, and amino acid; wherein, the cryoprotectant is present in a range of 9-91% of the composition.

6. The composition as claimed in claim 1, wherein the bulking agent is selected from the group consisting of sugar, polyol, polymer, and amino acid; wherein, the bulking agent is present in a range of 1-78% of the composition.

7. The composition as claimed in claim 1, wherein the buffer is selected from the group consisting of phosphate buffer, sodium phosphate buffer, potassium phosphate buffer, TRIS, and citrate buffer; wherein, the buffer is in a range of 3-33% of the composition.

8. The composition as claimed in claim 1, wherein the pharmaceutically acceptable excipient is present and is a salt.

9. The composition as claimed in claim 8, wherein the salt is selected from the group consisting of sodium chloride and potassium chloride; wherein an amount of the salt in the composition is in a range of up to 40% of the composition.

10. The composition as claimed in claim 1, wherein a pH before lyophilization and after reconstitution of the lyophilized composition is 6-8.

11. A process for preparation of the composition as claimed in claim 1, consisting of:
a. freezing;
b. optionally annealing;
c. primary drying; and
d. secondary drying.

12. The process as claimed in claim 11, wherein variation of temperature is in the range of from −60° C. to 30° C.

13. The process as claimed in claim 11, wherein a lowest temperature of the freezing step is from −10° C. to −60° C.

14. The process as claimed in claim 11, wherein the primary drying step is performed at a temperature from 10° C. to −50° C.

15. A process as claimed in claim 11, wherein the secondary drying step is performed at a temperature from 10° C.

16. A lyophilized composition obtained from the process as claimed in claim 11.

17. The composition as claimed in claim 1, wherein a final concentration of pegaspargase is in the range of 750±20% IU/ml when the composition is reconstituted post lyophilization to a volume per vial of 1-5.5 mL.
